(19) United States
(12) Patent Application Publication
(10) Pub. No.: US 2026/0158221 A1
(43) Pub. Date: Jun. 11, 2026

(54) COSMETIC FORMULA ATOMIZATION DEVICE WITH PISTON-SPRING STRUCTURE

(71) Applicant: L'Oreal, Paris (FR)

(72) Inventors: Wooram PARK, Shanghai (CN); Gong CHEN, Shanghai (CN)

(73) Assignee: L'Oreal, Paris (FR)

(21) Appl. No.: 19/124,155

(22) PCT Filed: Oct. 20, 2022
(86) PCT No.: PCT/CN2022/126379

§ 371 Date: Apr. 24, 2025

(30) Foreign Application Priority Data
Feb. 16, 2023 (FR) 20231447

(51) Int. Cl. A61M 5/30; A61M 5/20; A61M 5/31; A61M 5/315; A61M 11/06

(52) U.S. Cl. CPC A61M 5/3007; A61M 5/2033; A61M 5/315; A61M 11/06; A61M 2005/3143; A61M 2202/0468; A61M 2205/0216

(57) ABSTRACT

A system for atomizing and ejecting a formula including a tank, a nozzle fluidly connected to the tank including a Venturi tube and a tip, a cylinder with an outlet hole, the outlet hole fluidly connected to an inlet the Venturi tube, a piston arranged in the cylinder so as to be displaceable in the cylinder, a driving unit for the displacing of the piston in a direction in which a volume of an air intake space in the cylinder increases, an elastic member deformed according to the displacement of the piston and that stores an elastic energy while the piston is being displaced, where the driving unit includes an elastic energy release mechanism that releases the elastic energy, causing the piston to rush in and a vibration, and a dampener on the nozzle, positioned between the Venturi tube and the tip, where the dampener reduces the vibration.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

Cosmetic formulas, such as a liquid lotion including a hyaluronic acid, are typically applied to specific parts of the user's body, such as a face hands and arms, via a shallow transdermal delivery (i.e., a shallow depth skin delivery). For more effective transdermal delivery of such a formula, it is desirable to atomize the formula into micrometer-order fine droplets and to eject or spray it in a high velocity onto the desired part of the user's body. However, when the formula is atomized, it may result in a vibration or shockwave being delivered to the user, which may cause user discomfort.

In one aspect, the present disclosure describes a system for atomizing and ejecting a formula for a transdermal delivery, the system including a tank configured to hold the formula, a nozzle fluidly connected to the tank, comprising a tip and a Venturi tube comprising a longitudinal central axis and an internal passage extending along the longitudinal central axis where the internal passage is fluidly connected to the tank via an orifice defined in the Venturi tube, where the internal passage comprises a converging section, a diverging section, and a throat section, located between the converging section and the diverging section, a cylinder comprising a longitudinal central axis, an end wall, orthogonal to the longitudinal central axis; a circumferential wall extending from the end wall along the longitudinal central axis; and an outlet hold formed on the end wall, where the outlet hole is fluidly connected to an inlet of the converging section of the Venturi tube, a piston arranged in the cylinder and configured to be displaceable in the cylinder along the longitudinal central axis thereof, where the piston defines an air intake space in the cylinder in cooperation with the cylinder where the air intake space fluidly communicates with the inlet of the converging section of the Venturi tube via the outlet hole of the cylinder, a driving unit configured to displace of the piston in a direction in which a volume of the air intake space in the cylinder increases, an elastic member configured to be deformed according to the displacement of the piston and store an elastic energy therein while the piston is being displaced in the direction in which the volume of the air intake space increases, where the driving unit comprises an elastic energy release mechanism that releases the elastic energy stored in the elastic member, thereby causing the piston to rush in a direction in which the volume of the air intake space decreases, causing a vibration from the piston to the Venturi tube, and a dampener on the nozzle, positioned between the Venturi tube and the tip, where the dampener is configured to reduce the vibration.

In another aspect, an apparatus for transdermal delivery of an atomized formula, the apparatus including the system and a casing that at least partially houses the system disclosed.

In yet another aspect, a kit for transdermal delivery of an atomized formula, the kit including the apparatus and a plurality of nozzles, where the nozzle is a first nozzle of the plurality of nozzles, and where the plurality of nozzles is configured to couple to the apparatus is disclosed.

In another aspect, the present disclosure describes a method for atomizing and injecting a formula for a transdermal delivery, the method including filling a tank with the formula, where the tank is fluidly connected to a nozzle comprising a tip and a Venturi tube with an internal passage of the Venturi tube via an orifice defined in the Venturi tube, where the internal passage comprises a converging section, a diverging section, and a throat section located between the converging section and the diverging section, displacing a piston in a cylinder in a direction in which a volume of an air intake space in the cylinder increases, where the air intake space is defined between the piston and an end wall of the cylinder where an outlet hole formed on the end wall of the cylinder is fluidly connected to an inlet of the converging section of the Venturi tube, deforming, while the piston is being displaced in the direction in which the volume of the air intake space increases, an elastic member that is arranged so as to be deformed according to the displacement of the piston and stores an elastic energy therein, releasing the elastic energy stored in the elastic member, thereby causing the piston to rush in a direction in which the volume of the air intake space decreases, further causing a vibration from the piston to the Venturi tube, atomizing and ejecting to outside the formula supplied from the tank into the internal passage of the Venturi tube, by air pushed out from the cylinder through the outlet hole of the end wall of the cylinder with a rush of the piston, and reducing the vibration with dampener located between the Venturi tube and the tip of the nozzle.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a schematic block diagram of an example apparatus for transdermal delivery of an atomized liquid, in accordance with the present technology;

FIG. 2 is a schematic diagram of an example system for atomizing and ejecting a formula for a transdermal delivery, in accordance with the present technology;

FIG. 3 is an enlarged cross-sectional view of the Venturi tube of the example system in FIG. 2, in accordance with the present technology;

FIG. 4 is a perspective view of various gears forming a drive force transmission mechanism of a driving unit of the example system of FIG. 2, in accordance with the present technology;

FIG. 5 is an example apparatus, in accordance with the present technology;

FIG. 6 is an enlarged nozzle of the example apparatus of FIG. 5, in accordance with the present technology;

FIGS. 7A-7D showing the example apparatus of FIG. 5 in operation; in accordance with the present technology;

FIG. 8 is an example kit, in accordance with the present technology;

FIG. 9 illustrates a loadcell for measuring the vibration of an apparatus, in accordance with the present technology;

FIG. 10A is a graph showing the vibration of the apparatus without the dampener, in accordance with the present technology;

FIG. 10B is a graph showing the reduced vibration of the apparatus with the dampener, in accordance with the present technology; and

FIG. 11 is an example method of atomizing and ejecting a formula for transdermal delivery, in accordance with the present technology.

### DETAILED DESCRIPTION

While illustrative embodiments have been illustrated and described, it will be appreciated that various changes can be made therein without departing from the spirit and scope of the invention.

Described herein is a novel system for atomizing and ejecting a liquid (especially a liquid with a high molecular weight) for a transdermal delivery, as well as a novel apparatus for a transdermal delivery of a liquid, which includes such a system.

In particular, the present technology provides a novel system and apparatus for atomizing and ejecting a liquid for transdermal delivery, which do not require a replaceable, i.e., a single use/disposable power sources such as a high-pressure gas cartridge and can therefore atomize and can therefore atomize and eject the liquid as many times as the user desires. Furthermore, an object of the present technology is to provide a novel system and apparatus for atomizing and ejecting liquid for a transdermal delivery, which do not require an external supply source such as an air compressor or an air pump, and are therefore compact and easy to move, transport, and handle. Finally, the present technology provides features that provide shock absorbance/dampening to administer formula in a way that is comfortable to the user.

Furthermore, the system, the apparatus, and the method according to the present disclosure do not require a replaceable, i.e., single use/disposable motive power source such as a high-pressure gas cartridge in order to atomize and eject a liquid for a transdermal delivery. Therefore, the system, the apparatus, and the method according to the present technology can atomize and eject the liquid over and over again. In addition to this, the system, the apparatus, and the method according to the present technology do not require any external source such as an air compressor or an air pump. That is, a high-speed air jet for atomizing and discharging a liquid can be created internally. Therefore, the system and the apparatus can be compact as a whole enough to be handheld, and it is easy to move, transport and handle.

In some embodiments, the present technology includes a system for atomizing and ejecting a formula for a transdermal delivery, the system including a tank configured to hold the formula, a nozzle fluidly connected to the tank, comprising a tip and a Venturi tube comprising a longitudinal central axis and an internal passage extending along the longitudinal central axis where the internal passage is fluidly connected to the tank via an orifice defined in the Venturi tube, where the internal passage comprises a converging section, a diverging section, and a throat section, located between the converging section and the diverging section, a cylinder comprising a longitudinal central axis, an end wall, orthogonal to the longitudinal central axis; a circumferential wall extending from the end wall along the longitudinal central axis; and an outlet hold formed on the end wall, where the outlet hole is fluidly connected to an inlet of the converging section of the Venturi tube. In some embodiments, the system further includes a piston arranged in the cylinder and configured to be displaceable in the cylinder along the longitudinal central axis thereof, where the piston defines an air intake space in the cylinder in cooperation with the cylinder where the air intake space fluidly communicates with the inlet of the converging section of the Venturi tube via the outlet hole of the cylinder, a driving unit configured to displace of the piston in a direction in which a volume of the air intake space in the cylinder increases, an elastic member configured to be deformed according to the displacement of the piston and store an elastic energy therein while the piston is being displaced in the direction in which the volume of the air intake space increases, where the driving unit comprises an elastic energy release mechanism that releases the elastic energy stored in the elastic member, thereby causing the piston to rush in a direction in which the volume of the air intake space decreases, causing a vibration from the piston to the Venturi tube, and a dampener on the nozzle, positioned between the Venturi tube and the tip, where the dampener is configured to reduce the vibration.

In some embodiments, the dampener is selected from the group consisting of an elastic spring, a sponge, an air cushion, a rubber cushion, a bellows, an air cylinder damper, and combinations thereof. In some embodiments, the dampener includes an elastomer selected from the group consisting of rubber, nitrile rubber, fluororubber, silicon, thermoplastic elastomer (TPE), polyurethane, and combinations thereof. In some embodiments, the elastomer has a hardness of about 20-70 Shore A. In some embodiments, the elastomer has a hardness of about 30-60 Shore A.

In some embodiments, the system further includes a silencer disposed between the dampener and the tip and configured to further reduce the vibration. In some embodiments, the vibration is reduced to a force of about 0-20 gf.

In some embodiments, the piston includes a longitudinal central axis; an end wall facing the end wall of the cylinder; a circumferential wall extending from the end wall along the longitudinal central axis, where a rack extending along the longitudinal central axis is formed on an outer surface of the circumferential wall of the piston, where the driving unit comprises a sector gear having teeth only in a certain angle range, which meshes with the rack of the piston and drives it linearly, where the combination of the sector gear and the rack forms the elastic energy release mechanism.

In some embodiments, the driving unit further includes an electrical power supply; and an electric motor electrically connected to the electric power supply and configured to drive the sector gear rotationally directly or indirectly.

In some embodiments, the elastic member is a coil spring at least partially housed inside the piston. In some embodiments, the tank is fluidly connected to the throat section of the Venturi tube. In some embodiments, the formula is a cosmetic or aesthetical formula. In some embodiments, the formula may be a medication, a makeup, or a skin cream.

In another aspect, the present disclosure describes an apparatus for transdermal delivery of an atomized formula, the apparatus including the system described herein, and a casing that at least partially houses the system.

In yet another aspect, the present disclosure describes a kit for transdermal delivery of an atomized formula, the kit including the apparatus described herein, and a plurality of nozzles, where the nozzle is a first nozzle of the plurality of nozzles, and where the plurality of nozzles is configured to couple to the apparatus. In some embodiments, each nozzle of the plurality of nozzles comprises an outlet hole distinct in size from another nozzle of the plurality of nozzles. In some embodiments, each nozzle of the plurality of nozzles is configured to dispense a different formula. I n some embodiments, the plurality of nozzles is disposable.

In yet another aspect, the present disclosure describes a method for atomizing and injecting a formula for a transdermal delivery, the method including filling a tank with the formula, where the tank is fluidly connected to a nozzle comprising a tip and a Venturi tube with an internal passage of the Venturi tube via an orifice defined in the Venturi tube, where the internal passage comprises a converging section; a diverging section; and a throat section located between the converging section and the diverging section, displacing a piston in a cylinder in a direction in which a volume of an air intake space in the cylinder increases, where the air intake space is defined between the piston and an end wall of the cylinder where an outlet hole formed on the end wall of the cylinder is fluidly connected to an inlet of the converging section of the Venturi tube, deforming, while the piston is being displaced in the direction in which the volume of the air intake space increases, an elastic member that is arranged so as to be deformed according to the displacement of the piston and stores an elastic energy therein, releasing the elastic energy stored in the elastic member, thereby causing the piston to rush in a direction in which the volume of the air intake space decreases, further causing a vibration from the piston to the Venturi tube, atomizing and ejecting to outside the formula supplied from the tank into the internal passage of the Venturi tube, by air pushed out from the cylinder through the outlet hole of the end wall of the cylinder with a rush of the piston, and reducing the vibration with dampener located between the Venturi tube and the tip of the nozzle.

In some embodiments, the method further includes further reducing the vibration with a silencer disposed between the dampener and the tip of the nozzle. In some embodiments, the vibration is reduced to a force of about 0-20 gf.

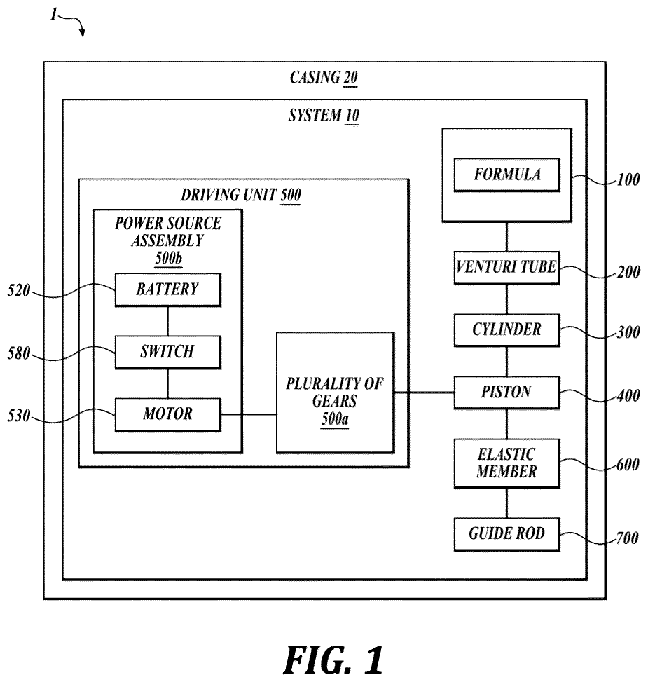

Some exemplary embodiments of the present technology are illustrated in FIGS. 1-8. In each figure, the scale ratio of width, length, height, diameter or the like of each element may not be constant and may be different from the actual parameter. It should be noted that in certain figures, certain elements or features are drawn larger or smaller than they are, for emphasis.

As used herein, the terms related to the direction such as “upper”, “lower”, “up”, “down”, “upward”, “downward”, “above”, “below”, “right”, “left” or the like should be understood in relation to the orientation of the system and the apparatus in the figures, which may or may not match the actual orientation in use. Furthermore, as is obvious to those skilled in the art, in this specification, the term “distal” or “distally” means a direction away from the Venturi tube that ejects or releases an atomized liquid. On the other hand, the term “proximal” or “proximally” means a direction closer to the Venturi tube.

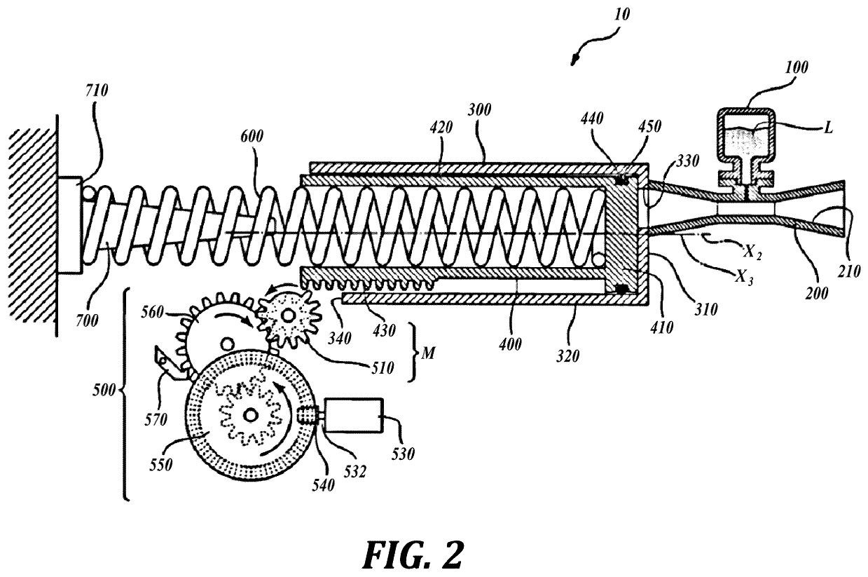

FIG. 1 is a schematic block diagram of an example apparatus for transdermal delivery of an atomized formula, in accordance with the present technology. This apparatus 1, may include a system 10, for atomizing and ejecting a liquid for a transdermal delivery, explained in detail herein, and a casing 20 that houses the system 10 therein. In the illustrated embodiment, the casing 20 approximately surrounds the entire system 10, except for an outlet of a Venturi tube for discharging the atomized liquid (as shown in FIG. 2), although the casing 20 may only house a portion of the system 10. The formula that the apparatus 1 targets for atomization and ejection is, in particular, a cosmetic formula such as a liquid lotion including a hyaluronic acid for transdermal delivery. However, the apparatus 1 and thus the system 10 may be used for an atomization and an ejection of water, oil, various lotions, and the like.

In addition, as illustrated in FIG. 2, the system 10 further comprises a tank 100 for holding a certain amount of formula (F), for example a few milliliters to a few hundred milliliters of a formula (F). The tank 100 is preferably made of, for example, a transparent plastic or glass so that a content and/or an amount of the formula can be seen from the outside. In some embodiments, translucent or opaque materials are used for making the tank 100. The system 10 also comprises a Venturi tube 200, a cylinder 300 disposed adjacent to the Venturi tube 200, and a piston 400 slidably arranged in the cylinder 300. The Venturi tube 200 may be formed from a plastic material having a sufficient rigidity, for example, acrylonitrile butadiene styrene (ABS), polypropylene (PP), polycarbonate (PC) and the like. The cylinder 300 and the piston 400 may also be formed from the same material. In some embodiments, both the cylinder 300 and the piston 400 are formed from a suitable metal (or metal alloy) material.

The system 10 additionally may include a driving unit 500 that is operatively (i.e., mechanically) associated with the piston 400, an elastic member 600 (such as a coil spring) that is also mechanically associated with the piston 400, and an elongated guide rod 700 arranged to be surrounded by the coil spring 600.

Particularly, as shown in FIG. 1, the driving unit 500 may mainly comprises a group of gears 500a and a power source assembly 500b, described in detail herein. In this embodiment, the power source assembly 500b includes a battery (or electric power supply) 520, a motor 530 (or electric motor), and a switch 580 interposed therebetween. It should be noted that the battery 520 and the switch 580 are not shown in figures other than FIG. 1 for convenience.

FIG. 2 is a schematic diagram of an example system for atomizing and ejecting a formula for a transdermal delivery, in accordance with the present technology. In some embodiments, the system (such as system 1) includes a cylinder 300, a piston 400.

In some embodiments, the cylinder 300 includes a longitudinal central axis X₂, an end (proximal end) wall 310 facing the Venturi tube 200 and orthogonal to the longitudinal central axis X₂, and a circumferential wall 320 extending from the end wall 310 along the longitudinal central axis X₂. That is, the cylinder 300 is a hollow body with one end open. In some embodiments, the cylinder 300 is fixedly supported by a frame of the system 10 (for example, a subcase (not shown) of the apparatus 1). The cylinder 300 further comprises an outlet hole 330 formed on the end wall 310 thereof. The outlet hole 330 is fluidly connected to the inlet 232 of the converging section 230 of the Venturi tube 200. In this embodiment, the Venturi tube 200 is directly connected to the cylinder 300 such that the inlet 232 of the converging section 230 of the Venturi tube 200 and the outlet hole 330 of the cylinder 300 are aligned with each other. However, it is also possible to adopt a configuration in which the inlet 232 of the converging section 230 and the outlet hole 330 of the cylinder 300 are connected via a pipeline or a conduit line.

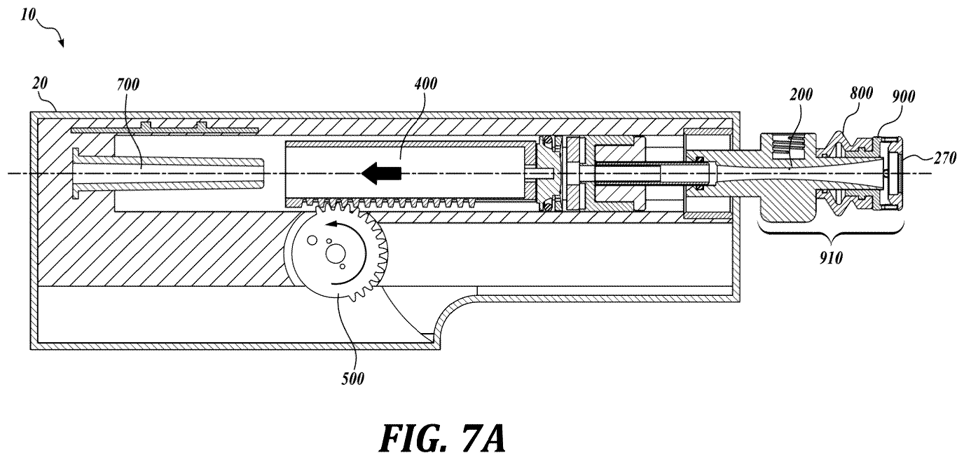

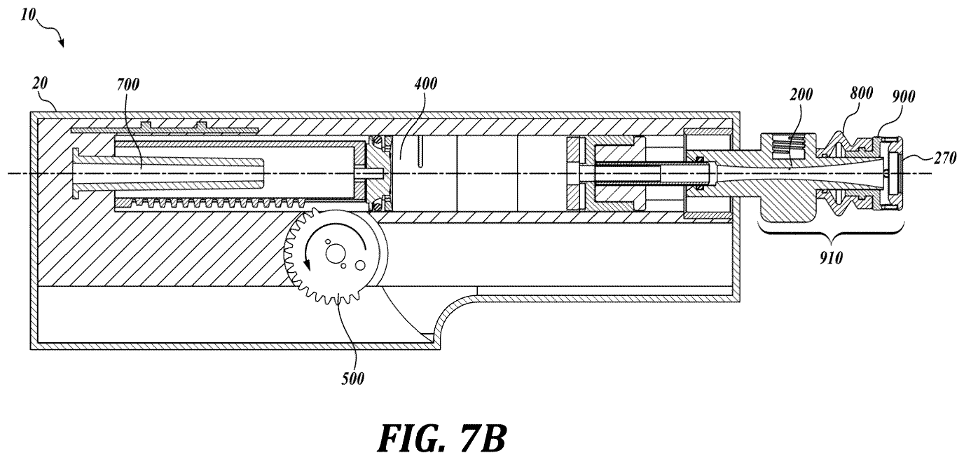

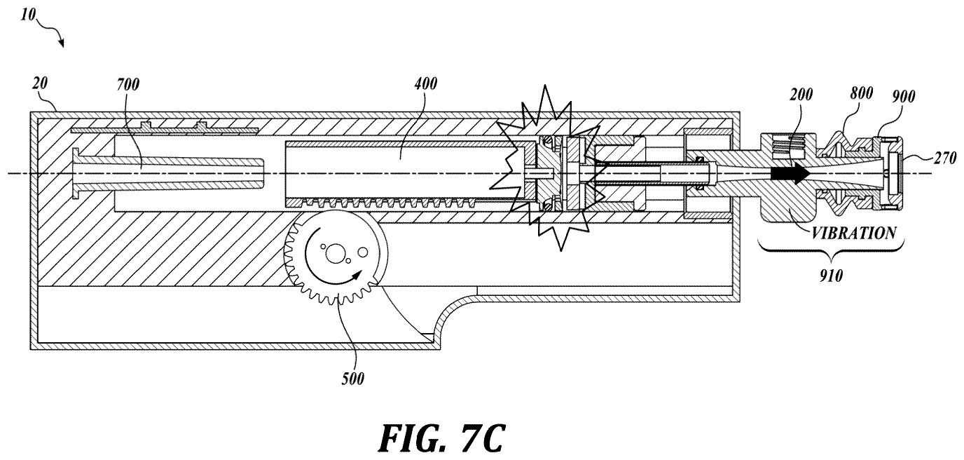

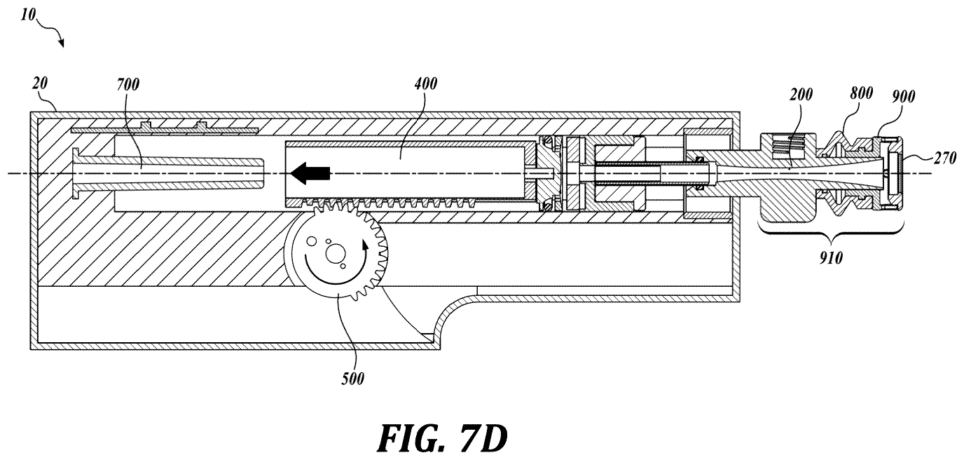

In the cylinder 300, the piston 400, is arranged so as to be smoothly displaceable along the longitudinal central axis X₂ of the cylinder 300. The piston 400 comprises a longitudinal central axis X₃, an end (proximal end) wall 410 facing the end wall 310 of the cylinder 300, and a circumferential wall 420 extending from the end wall 410 along the longitudinal central axis X₃. Furthermore, the piston 400 may include a rack 430 extending along the longitudinal central axis X₃. In some embodiments, the rack 430 is integrally formed on an outer surface of the circumferential wall 420 of the piston 400, in particular, in approximately half the area on the distal end side of the circumferential wall 420. Although not shown in FIG. 2, in some embodiments, the piston 400 defines an air intake space V in the cylinder 300 in cooperation with it (as shown in FIG. 7A-7D). In some embodiments, the air intake space V fluidly communicates with the inlet 232 of the converging section 230 of the Venturi tube 200 via the outlet hole 330 of the cylinder 300.

The piston 400 may further include an O-ring (packing) 440 made of, for example, an elastomeric material. The O-ring 440 is fitted in a circular groove 450 formed in the end wall 410 of the piston 400. The O-ring 440 serves to maintain airtightness between the piston 400 and the inner surface of the cylinder 300. In this embodiment, the cross section of the piston 400 is a perfect circle in its end wall section where the O-ring 440 is located. However, in some embodiments, in the circumferential wall section, the cross section of the piston 400 is a partial circle in which a part of the circle is cut off along a straight line parallel to its diameter. This is to secure a flat surface on the outer peripheral surface of the piston for arranging the rack 430 as described above. In other embodiments, other shapes may be adopted as the cross section of the circumferential wall section of the piston 400. Although not shown, in this embodiment, the system 10 further includes a mechanism or feature for preventing the piston 400 from rotating with respect to the cylinder 300.

In the state shown in FIG. 2, that is, the state in which the end wall 310 of the cylinder 300 and the end wall 410 of the piston 400 are in contact with each other, a distal end of the piston 400 may protrude somewhat from the distal end of the cylinder 300. In other words, the length of the piston 400 along the longitudinal central axis X₃ may be somewhat greater than the length of the circumferential wall 320 of the cylinder 300 along the longitudinal central axis X₂. Therefore, in the state shown in FIG. 2, a part of the rack 430 integrally formed on the outer peripheral surface of the piston 400 protrudes from the distal end of the cylinder 400. In this embodiment, the circumferential wall 320 of the cylinder 300 is formed with a linear notch 340 for exposing at least a part of the rack 430 of the piston 400. As explained in detail herein, in some embodiments, a sector gear 510 of the driving unit 500 meshes with the rack 430 through this notch 340 of the circumferential wall 320 of the cylinder 300.

The system 10 includes the driving unit 500 and the elastic member 600 mechanically associated with each other. The driving unit 500 may be configured to displace the piston 400 distally, i.e., in a direction in which a volume of the air intake space V in the cylinder 300 increases. On the other hand, the elastic member 600 may be arranged to be deformed according to the displacement of the piston 400 and stores an elastic energy (mechanical potential energy) therein while the piston 400 is being displaced distally, i.e., in the direction in which the volume of the air intake space V increases. Therefore, the system 10 of this embodiment can be referred to as a “spring-loaded system”. In this embodiment, the elastic member 600 is a coil spring, which is housed inside the hollow piston 400 at least in part, for example, about half.

The driving unit 500 additionally may include an elastic energy release mechanism M. In this embodiment, the elastic energy release mechanism M is configured to release, at a regular interval, the elastic energy stored in the elastic member 600, thereby causing the piston 400 to rush (or dash) proximally, i.e., in a direction in which the volume of the air intake space V decreases. More specifically, the driving unit 500 comprises a sector gear 510 having teeth only in a certain angle range, such as, for example 90° to 300°. The sector gear 510 is arranged to mesh with the rack 430 of the piston 400 and drives it linearly in one direction (i.e., to the left side in the figure). In this embodiment, the combination of the sector gear 510 and the rack 430 forms the elastic energy release mechanism M for releasing a stored elastic energy at a regular interval. That is, at the moment when the last tooth of the sector gear 510 disengages from the last tooth of the rack 430, the restraint of the piston 400 is released, and thus the elastic energy stored in the elastic member 600 is also released instantly. However, another type of the elastic energy release mechanism may be adapted and may be configured to release the elastic energy stored in the elastic member 600 only when desired. In some embodiments, as described in detail in FIGS. 7A-7D, the rushing of the piston 400 generates a vibration (or shock wave) from the piston 400 to the Venturi tube (200).

In some embodiments, the driving unit 500 comprises the electric power supply 520, for example, rechargeable batteries such as lithium-ion batteries, and the electric motor 530. In some embodiments, the electric motor 500 is electrically connected to the electric power supply 520 and indirectly (that is, via a gear train for a power transmission) drives the sector gear 510 rotationally. The electric power supply 520, the electric motor 530, and the switch 580 interposed therebetween constitute the power source assembly 500b of the driving unit 500 as described herein. In this embodiment, the sector gear 510 is rotated by the electric motor 530 via the group of gears 500a in only one direction, that is, counterclockwise in FIG. 2. In another embodiment, the sector gear 510 may be driven directly by the electric motor 530. However, in this case, since a motor having a large torque and therefore a large size is required, it is desirable to drive the sector gear 510 via a suitable reduction mechanism consisting of a gear train, as illustrated herein.

The driving unit 500 may further include a latch 570 that meshes with the spur gear 560. In some embodiments, the latch 570 is arranged so as to regulate a direction of rotation of the spur gear 560 so that the spur gear 560 rotates in only one direction (i.e., clockwise in FIG. 2). In another embodiment, the latch 570 is engaged with any other gear than the spur gear 560. In some embodiments, the driving unit 500 does not include the latch 570.

In some embodiments, the system 10 additionally includes an elongated spring guide rod 700, which is arranged so as to be surrounded by the coil spring 600. In this embodiment, a base end 710 of the guide rod 700 is supported by the frame (not shown) of the system 10 (i.e., a subcase of apparatus 1). In another embodiment, the spring guide rod 700 may be supported by the casing 20 itself of the apparatus 1. The spring guide rod 700 is arranged such that it at least partially enters the piston 400 when the piston 400 is being displaced distally, i.e., in the direction in which the volume of the air intake space V increases.

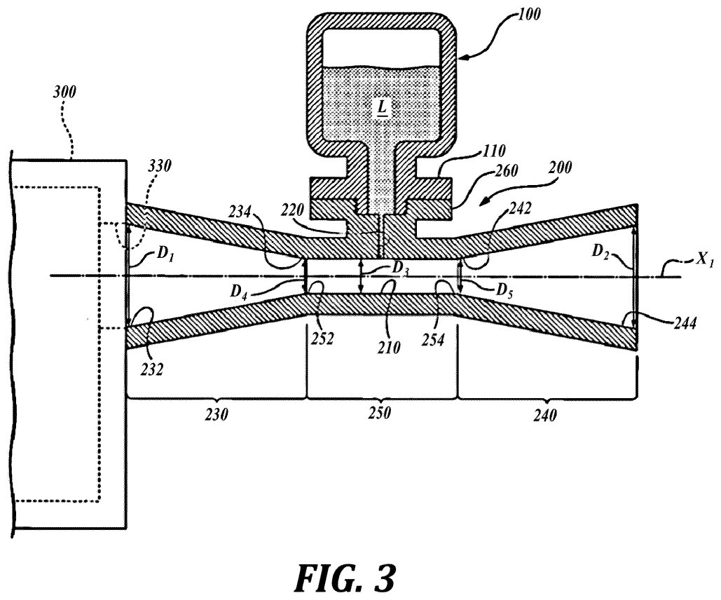

FIG. 3 is an enlarged cross-sectional view of the Venturi tube of the example system in FIG. 2, in accordance with the present technology. In some embodiments, the system 10 includes the tank 100 for holding the formula (or liquid) L. In this embodiment, the tank 100 is detachably connected in a liquid-tight manner to the Venturi tube 200 by way of screwing (as shown in FIG. 3).

In some embodiments, the tank 100 has a flange 110 on the opening side thereof. The Venturi tube 200 may also have a flange 260, corresponding to the flange 110 of the tank 100. In some embodiments, the tank 100 is placed above the Venturi tube 200 with its flange 110 in contact with the flange 260 of the Venturi tube 200. This arrangement is particularly preferred as the action of gravity facilitates the supply of formula L into the Venturi tube 200 during the system 10 operates. However, the orientation of the tank 100 with respect to the Venturi tube 200 is not limited to this and can be appropriately changed as needed.

In some embodiments, the Venturi tube 200 includes a longitudinal central axis X₁ and an internal passage 210 continuously extending along the longitudinal central axis X₁. In some embodiments, the internal passage 210 is fluidly connected to the tank 100 via an orifice 220 defined in the Venturi tube 200. As shown in FIG. 3, the internal passage 210 may include a converging section 230, a diverging section 240, and a throat section 250, which are connected continuously along the longitudinal central axis X₁. The throat section 250 may be located between the converging section 230 and the diverging section 240. In some embodiments, the tank 100 is fluidly connected to the throat section 250 of the Venturi tube 200 via the orifice 220 as explained above. The inner diameter of the orifice 220 may be configured so that when the pressure in the Venturi tube 200 is equal to the atmospheric pressure, the formula L does not spontaneously drop in the Venturi tube 200 due to the viscosity of the formula L.

In some embodiments, the converging section 230 has an inlet 232 and an outlet 234 placed on each of two ends thereof. The throat section 250 also has an inlet 252 and an outlet 254 placed on each of two ends thereof. Furthermore, the diverging section 240 has an inlet 242 and an outlet 244 placed on each of two ends thereof. The outlet 234 of the converging section 230 and the inlet 252 of the throat section 250 connects with each other smoothly and continuously. Similarly, the outlet 254 of the throat section 250 and the inlet 242 of the diverging section 240 connects with each other smoothly and continuously. In some embodiments, the inner diameter D₃ of the throat section 250 is constant. In some embodiments, the inner diameter (minimum inner diameter) D₄ of the outlet 234 of the converging section 230 is the same as the inner diameter D₃ of the throat section 250, and the inner diameter (minimum inner diameter) D₅ of the inlet 242 of the diverging section 240 is also the same as the inner diameter D₃ of the throat section 250. In some embodiments, the inner diameter of the converging section 230 decreases monotonically (linearly) towards the throat section 250, while the inner diameter of diverging section 240 increases monotonically (linearly) away from the throat section 250. However, the inner diameters of the converging and diverging sections 230, 240 may curvilinearly decrease and increase, respectively.

In some embodiments, the ratio of the maximum inner diameter D₁ (i.e., the inner diameter of the inlet 232) of the converging section 230, the inner diameter D₃ of the throat section 250, and the maximum inner diameter D₂ (i.e., the inner diameter of outlet 244) of the diverging section 240, i.e., D₁:D₃:D₂, is 1:0.1 to 0.7:1 to 1.5. This is based on the maximum inner diameter D₁ of the converging section 230. However, this ratio is just an example, and various other ratios can be adopted as needed.

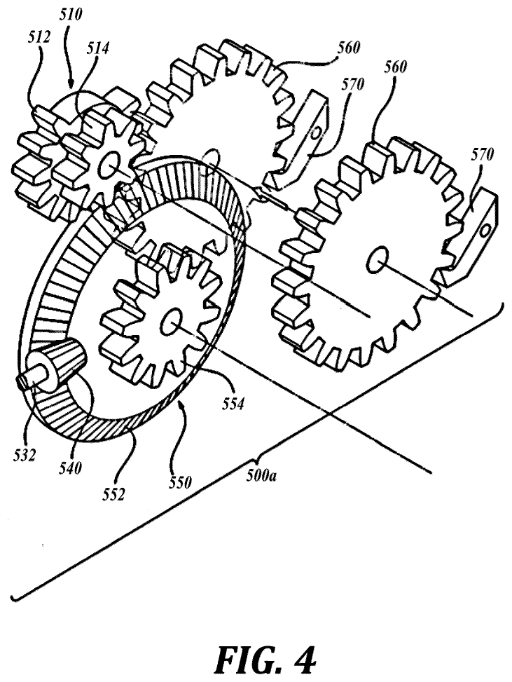

FIG. 4 is a perspective view of various gears forming a drive force transmission mechanism of a driving unit 500 of the example system of FIG. 2, in accordance with the present technology. In some embodiments, the driving unit 500 includes a group of gears 500a. In some embodiments, the gear train including a reduction mechanism of the driving unit 500, i.e., the group of gears 500a of the driving unit 500, is disclosed herein. The group of gears 500a of the driving unit 500 may include a pinion 540 (of a bevel gear type, a worm gear type, a spur gear type, etc.) fixedly coupled to an output shaft 532 of the electric motor 530. Furthermore, the group of gears 500a of the driving unit 500 may further include two kinds of gears 550, 560 that transmit a rotational motion of the pinion 540 to the sector gear 510 to rotationally drive it. In such embodiments, these gears 550, 560 as well as the sector gear 510 are rotatably supported by the frame (not shown) of the system 10 (i.e., a subcase of the apparatus 1). In such embodiments, since the electric motor 530 is fixedly supported by the frame (not shown) of the system 10, the pinion 540 is also rotatably supported by the frame (not shown) of the system 10. In another embodiment, the gears 510, 550, and 560 may be rotatably supported by the casing 20 itself of the apparatus 1. In some embodiments, the gear 550 that meshes with the pinion 540 is a bevel gear. In other embodiments, the gear 560 that meshes with both the bevel gear 550 and the sector gear 510 is a spur gear.

In other embodiments, the spur gear 560 functions as an intermediate gear. In such embodiments, both the sector gears 510 and the bevel gear 550 have a structure in which two types of gearing portions are stacked along the direction of rotational axis. The sector gear 510 may include a first portion 512 with teeth only in a certain angular range along the root circle thereof. Furthermore, the sector gear 510 may include a second portion 514 integrally coupled to this first portion 512. In some embodiments, the second portion 514 of the sector gear 510, i.e., a spur gear portion, includes teeth along the entire circumference of the root circle thereof. In some embodiments, the diameter of the addendum circle of the second portion 514 is smaller than that of the first portion 512.

In some embodiments, the bevel gear 550 also includes a first portion 552 containing a row of teeth arranged circumferentially on a conical surface, and a second portion 554 integrally coupled to this first portion 552 and consisting of a spur gear smaller in diameter than the minimum diameter of the first portion 552. In some embodiments, the pinion 540 fixedly attached to the output shaft 532 of the electric motor 530 meshes with the first portion 552 of the bevel gear 550, and the second portion 554 of the bevel gear 550 that rotates integrally with it meshes with the spur gear 560. Further, in some embodiments, the spur gear 560 meshes with a second portion 514 of the sector gear 510.

As a result, in operation, the high-speed rotation of the pinion 540 causes the sector gear 510 to rotate at a predetermined lower speed, for example, several revolutions per second. The piston 400 is displaced in the distal direction at a regular interval by the rotation of the sector gear 510 thus resulting. In this embodiment, the number of teeth on the rack 430 of the piston 400 is approximately equal to the number of teeth on the first portion 512 of the sector gear 510, but the present technology is not limited thereto.

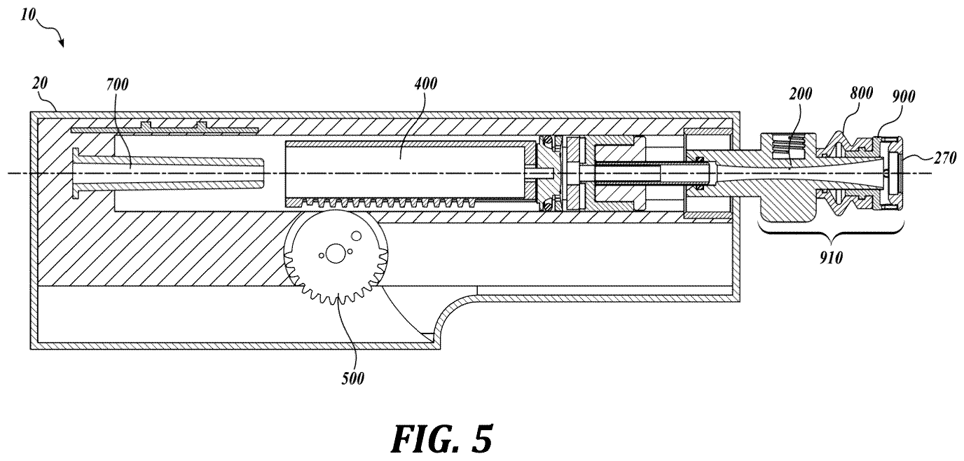

FIG. 5 is an example apparatus 10, in accordance with the present technology. In some embodiments, the apparatus 10 includes a casing 20, a piston 400, a Venturi tube 200, a dampener 800, a silencer 900, and a tip 270. It should be understood that the Venturi tube 200 and the tip 200 can be referred to collectively as a nozzle 910. In some embodiments, the nozzle 910 also includes the dampener 800 and the silencer 900. While FIG. 5 illustrates an apparatus 10 including a casing 20, it should be understood that the apparatus may not include a casing 20.

In some embodiments, the apparatus 10 includes a dampener 800 configured to reduce a vibration generated by the piston 400, as described in FIGS. 7A-7D. In some embodiments, the dampener 800 is in a bellows shape 800 as shown in FIG. 5. However, it should be understood that the dampener can take any number of forms, including, but not limited to an elastic spring, a sponge, an air cushion, a rubber cushion, an air cylinder damper, and combinations thereof. In some embodiments, the dampener 800 is made of an elastomer. In some embodiments, the dampener is made out of rubber, nitrile rubber, fluororubber, silicon, thermoplastic elastomer (TPE), polyurethane, and combinations thereof. In some embodiments, the dampener is located between the Venturi tube 200 and the tip 270 of the nozzle 910. In some embodiments, the dampener may be located elsewhere on the apparatus 10, including but not limited to the tip 270, and between the venturi tube 200 and the casing 20. In some embodiments, the elastomer has a hardness of about 20-70 Shore A. In some embodiments, the elastomer has a hardness of about 30-60 Shore A. In operation, the dampener absorbs a vibration generated by the piston when the piston rushes forward, as described in further detail FIGS. 7A-7D.

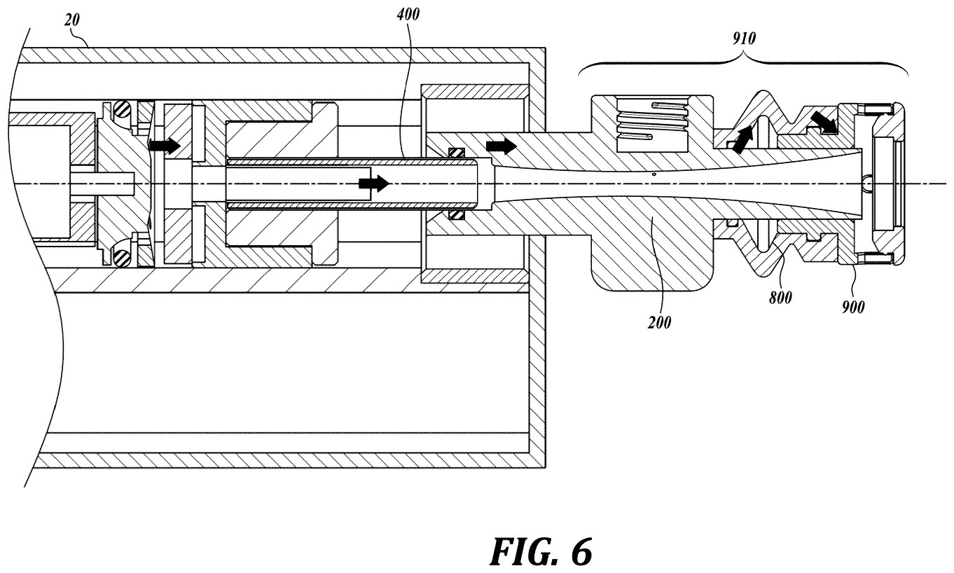

FIG. 6 is an enlarged nozzle 910 of the example apparatus 10 of FIG. 5, in accordance with the present technology. In some embodiments, the apparatus 10 includes a casing 20, a piston 400, a nozzle 910 including a Venturi tube 200 and a tip 270, an elastomer 800 and a silencer 900. The arrows in FIG. 6 indicate how the vibration (shockwave) moves throughout the apparatus when the piston rushes forward in the direction in which the volume of air intake space in the cylinder increase, as shown in more detail in FIGS. 7A-7D.

FIGS. 7A-7D showing the example apparatus 10 of FIG. 5 in operation, in accordance with the present technology. In some embodiments, the apparatus includes a casing 20, a driving unit 500, which may include any number of gears, as illustrated in FIG. 4, a piston 400, and a nozzle 910 including a Venturi tube and a dampener 800, and an elongated guide rod 700. It should be understood that an elastic member (such as elastic member 600) may also be included in apparatus 10 but is omitted here for clarity.

In each of FIGS. 7A-7D, it should be understood that a tank (such as tank 100 in FIGS. 2 and 3) may be attached to the Venturi tube 200 as shown in FIGS. 2 and 3. In operation, the tank is filled with the formula. In some embodiments, the tank is fluidly connected to the nozzle 910, which includes the Venturi tube 200. In some embodiments, the tank is connected to the nozzle 910 through an internal passage (such as shown in FIG. 3) of the Venturi tube 200 via an orifice defined in the Venturi tube 200.

In FIG. 7A, the piston 400 in a cylinder in a direction in which a volume of an air intake space in the cylinder increases, as indicated by the arrow in FIG. 7A. In some embodiments, the air intake space is defined between the piston 400 and an end wall of the cylinder where an outlet hole formed on the end wall of the cylinder is fluidly connected to an inlet of the converging section of the Venturi tube 200.

As shown in FIG. 7B, while the piston is being displaced in the direction in which the volume of the air intake space increases, an elastic member (such as elastic member 700 of FIG. 2) is deformed according to the displacement of the piston and stores an elastic energy therein. In some embodiments, the elastic member is a spring.

In FIG. 7C, the elastic energy stored in the elastic member is released, thereby causing the piston 400 to rush in a direction in which the volume of the air intake space decreases, further causing a vibration from the piston to the Venturi tube. The arrow represents the direction of the vibration, while the burst illustrates the impact of the piston 400. It should be understood that as the piston rushes forward, the formula is atomized and ejected to outside the formula supplied from the tank into the internal passage of the Venturi tube, by air pushed out from the cylinder through the outlet hole of the end wall of the cylinder with a rush of the piston 400. The vibration is reduced with the dampener 800 located between the Venturi tube 200 and the tip of the nozzle 910.

In FIG. 7D, the piston 400 returns to its initial position and the process starts over. In some embodiments, a user can determine when the apparatus 10 atomizes and dispenses the formula, either through an actuator on the apparatus, or through an application communicatively coupled to the apparatus 10. In some embodiments, the user can manually determine when the apparatus 10 atomizes and dispenses the formula, such as through a trigger or a slide wheel on the apparatus. In some embodiments, by actuating an actuator a single time, the apparatus 10 will continue to move through the states illustrated in FIGS. 7A-7D until the actuator is actuated for a second time.

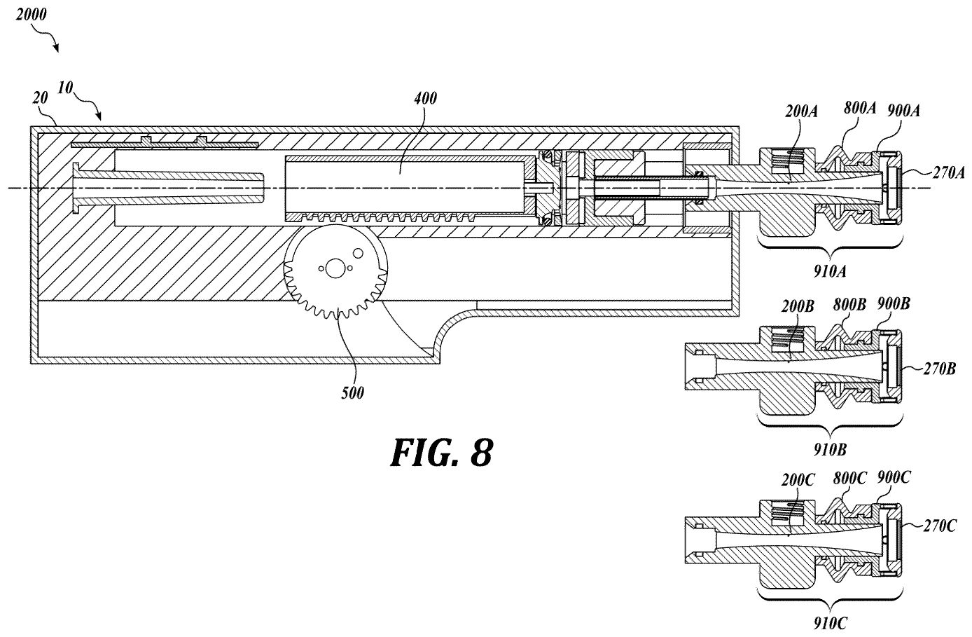

FIG. 8 is an example kit 2000, in accordance with the present technology. In some embodiments, the apparatus 10 is a part of a larger kit 2000.

In some embodiments, the nozzle 910A is a first nozzle. In some embodiments, the kit 2000 includes a plurality of nozzles 910A, 910B, and 910C. In some embodiments, each nozzle 910 includes a Venturi tube, a tip, a dampener, and a silencer. In some embodiments, some nozzles 910 may include the dampener and silencer, while others do not. In some embodiments, the nozzles 910A, 910B, and 910C are sized distinctly from one another. In such embodiments, each nozzle of the plurality of nozzles 910A, 910B, 910C is configured to dispense a different formula. In some embodiments, each nozzle of the plurality of nozzles 910A, 910B, 910C are replacements for one another. In these embodiments, each nozzle 910 is the same size. In some embodiments, the outlet of each nozzle of the plurality of nozzles 910A, 910B, 910C is sized differently, to better dispense different formulas of different consistencies or viscosities.

In some embodiments, each nozzle of the plurality of nozzles 910A, 910B, 910C is disposable, but in other embodiments, each nozzle of the plurality of nozzles 910A, 910B, 910C is reusable.

### EXAMPLE

The apparatus (as illustrated in FIG. 5) having both the dampener 800 and the silencer 900 was tested to determine whether the vibration (or shock wave) was dampened, and to what degree.

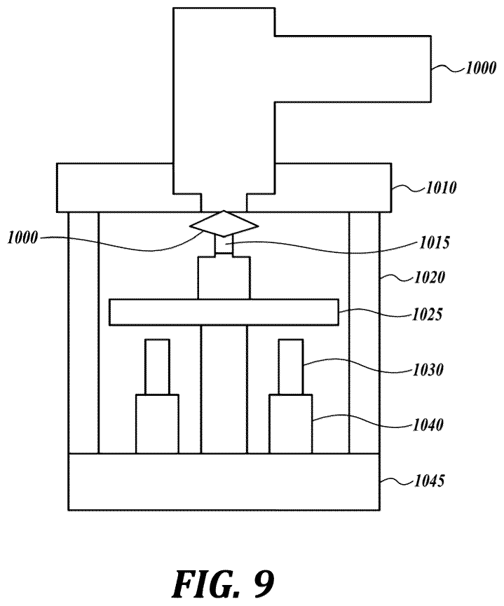

FIG. 9 illustrates a loadcell for measuring the vibration of an apparatus, in accordance with the present technology.

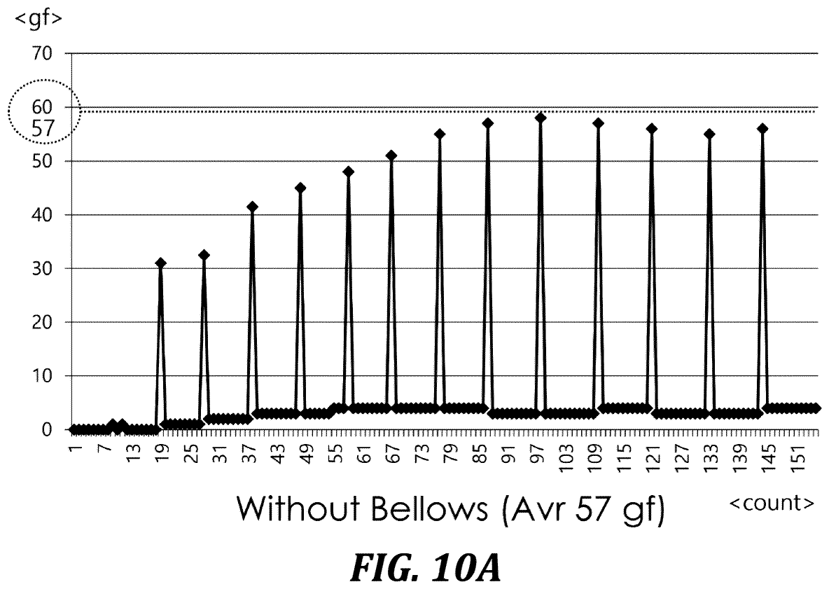

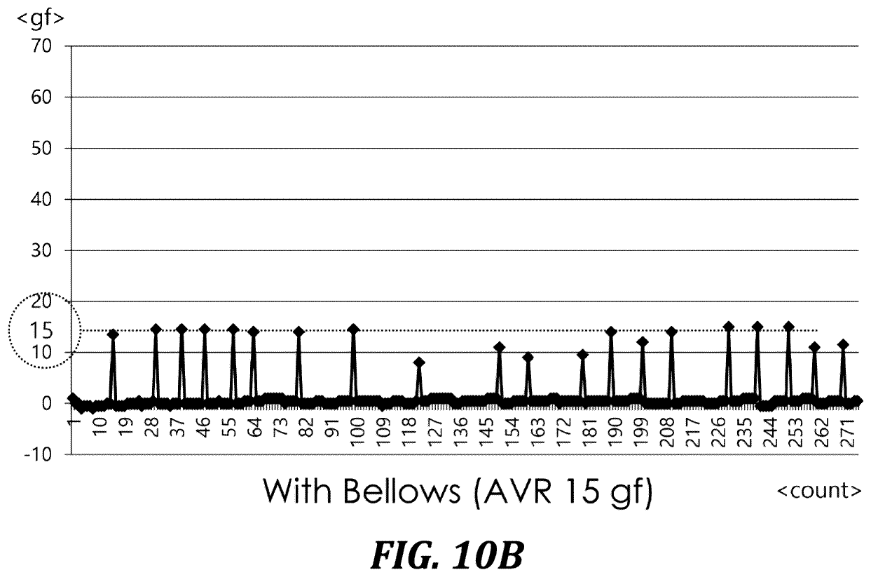

In order to test this, a testing device including a holder 1010, a bracket 1020, and a base plate 1045 was prepared. The testing device also included a load cell plate 1025, a first loadcell stopper 1030, a second stopper 1040. A loadcell 1035 was placed below the load cell plate and on top of the base plate 1045. An apparatus 1000, as described herein, was placed on the testing device as illustrated in FIG. 9. The shock energy (or vibration) of the apparatus 1000 was measured by the testing device during continuous piston motion both without the dampener 1000 and silencer 1015 (FIG. 10A) and with the dampener 1000 and silencer 1015 (FIG. 10B).

FIG. 10A is a graph showing the vibration of the apparatus without the dampener, in accordance with the present technology. On the horizontal axis is the number of the reading of the load cell/sensor. 100 means 100 times of reading, approximately 15 pulse readings per second. On the vertical axis is the force in gram-force (gf). Each spike represents the piston rushing forward, as described in FIGS. 7A-7D. The dashed vertical line labeled “57” indicates where 57 gf is in relation to each piston thrust.

As shown in FIG. 10A, without the dampener, the average force was 57 gf. Further, even when the piston was not actively rushing forward, the apparatus 1000 exerted force on the loadcell.

FIG. 10B is a graph showing the reduced vibration of the apparatus with the dampener, in accordance with the present technology. On the horizontal axis is the number of the reading of the load cell/sensor. 100 means 100 times of reading, approximately 15 pulse readings per second. On the vertical axis is the force in gram-force (gf). Each spike represents the piston rushing forward, as described in FIGS. 7A-7D. The dashed vertical line labeled “57” indicates where 15 gf is in relation to each piston thrust.

As shown in FIG. 10B, when the dampener was attached to the apparatus, the force (or vibration) of the apparatus was greatly reduced. The average force of the apparatus with the dampener was only 15 gf. Additionally, when the piston was not rushing forward, the overall vibration or force of the apparatus was also reduced.

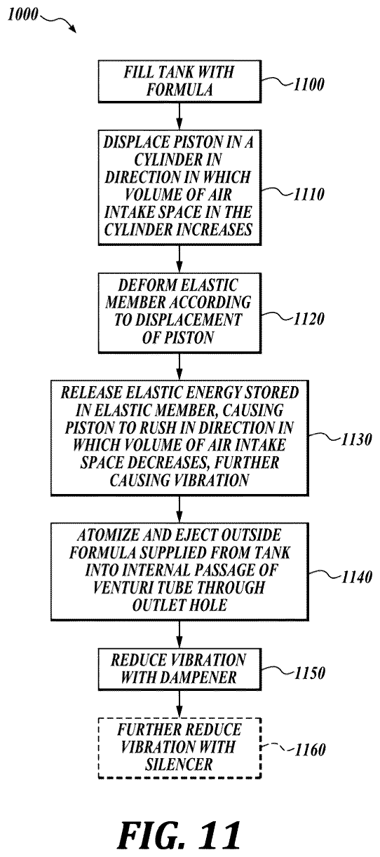

FIG. 11 is an example method of atomizing and ejecting a formula for transdermal delivery, in accordance with the present technology.

In block 1100, the tank is filled with formula. In some embodiments, the formula is a liquid. In some embodiments, the formula is a cosmetic formula. In some embodiments, the formula is a liquid lotion.

In block 1110, the piston is displaced inside the cylinder in the direction in which the volume of air intake space in the cylinder increase.

In block 1120, the elastic member is deformed in response to the displacement of the piston. In some embodiments, the elastic member stores elastic energy when it is deformed. In some embodiments, the elastic member is a spring. In such embodiments, the spring compresses in response to the piston being displaced.

In block 1130, the elastic energy stored in the elastic member is released. In some embodiments, in response to this elastic energy, the piston rushes in the direction in which the volume of air intake space decreases. When the piston rushes forward, in some embodiments, a vibration (or shockwave) is caused from the piston to the Venturi tube.

In block 1140, the formula is atomized and ejected from the tank and into the internal passage of the Venturi tube, through the outlet hole. In some embodiments, the atomized formula is ejected onto a user's skin or hair.

In block 1150, the vibration from the piston to the Venturi tube (or the nozzle) us dampened with the dampener on the nozzle, as described herein. As the Venturi tube moves in response to the piston rushing forward, the dampener absorbs at least a part of the vibration. In some embodiments, this improves the user experience by minimizing discomfort as the nozzle contacts the user's skin, face, or hair. In some embodiments, the vibration is reduced to a force of about 0-20 gf.

Optionally, in block 1160, the vibration is further reduced by the silencer as described herein. In some embodiments, the silencer further improves the user experience by minimizing user discomfort.

The order in which some or all of the blocks in the method should not be deemed to be limiting. Rather, one or ordinary skill in the art having the benefit of the present disclosure will understand that some of the blocks may be executed in a variety of orders not illustrated, or even in parallel.

The detailed description set forth above in connection with the appended drawings, where like numerals reference like elements, are intended as a description of various embodiments of the present disclosure and are not intended to represent the only embodiments. Each embodiment described in this disclosure is provided merely as an example or illustration and should not be construed as preferred or advantageous over other embodiments. The illustrative examples provided herein are not intended to be exhaustive or to limit the disclosure to the precise forms disclosed. Similarly, any steps described herein may be interchangeable with other steps, or combinations of steps, in order to achieve the same or substantially similar result. Generally, the embodiments disclosed herein are non-limiting, and the inventors contemplate other embodiments within the scope of this disclosure may include structures and functionalities from more than one specific embodiment shown in the figures and described in the specification.

In the foregoing description, specific details are set forth to provide a thorough understanding of exemplary embodiments of the present disclosure. It will be apparent to one skilled in the art, however, that the embodiments disclosed herein may be practiced without embodying all the specific details. In some instances, well-known process steps have not been described in detail in order not to unnecessarily obscure various aspects of the present disclosure. Further, it will be appreciated that embodiments of the present disclosure may employ any combination of features described herein.

The present application may include references to directions, such as “vertical,” “horizontal,” “front,” “rear,” “left,” “right,” “top,” and “bottom,” etc. These references, and other similar references in the present application, are intended to assist in helping describe and understand the particular embodiment (such as when the embodiment is positioned for use) and are not intended to limit the present disclosure to these directions or locations.

The present application may also reference quantities and numbers. Unless specifically stated, such quantities and numbers are not to be considered restrictive, but exemplary of the possible quantities or numbers associated with the present application. Also in this regard, the present application may use the term “plurality” to reference a quantity or number. In this regard, the term “plurality” is meant to be any number that is more than one, for example, two, three, four, five, etc. The term “about,” “approximately,” etc., means plus or minus 5% of the stated value. The term “based upon” means “based at least partially upon.”

The principles, representative embodiments, and modes of operation of the present disclosure have been described in the foregoing description. However, aspects of the present disclosure, which are intended to be protected, are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the present disclosure as claimed.

The embodiments of the invention in which an exclusive property or privilege is claimed are defined as follows:

1. A system for atomizing and ejecting a formula for a transdermal delivery, the system comprising:
a tank configured to hold the formula;
a nozzle fluidly connected to the tank, comprising a tip and a Venturi tube comprising a longitudinal central axis and an internal passage extending along the longitudinal central axis wherein the internal passage is fluidly connected to the tank via an orifice defined in the Venturi tube, wherein the internal passage comprises a converging section, a diverging section, and a throat section, located between the converging section and the diverging section;
a cylinder comprising a longitudinal central axis, an end wall, orthogonal to the longitudinal central axis; a circumferential wall extending from the end wall along the longitudinal central axis; and an outlet hold formed on the end wall, wherein the outlet hole is fluidly connected to an inlet of the converging section of the Venturi tube;
a piston arranged in the cylinder and configured to be displaceable in the cylinder along the longitudinal central axis thereof, wherein the piston defines an air intake space in the cylinder;
a driving unit configured to displace of the piston in a direction in which a volume of the air intake space in the cylinder increases,
an elastic member configured to be deformed according to the displacement of the piston and store an elastic energy therein while the piston is being displaced in the direction in which the volume of the air intake space increases, wherein the driving unit comprises an elastic energy release mechanism that releases the elastic energy stored in the elastic member, thereby causing the piston to rush in a direction in which the volume of the air intake space decreases, causing a vibration from the piston to the Venturi tube; and
a dampener on the nozzle, positioned between the Venturi tube and the tip, wherein the dampener is configured to reduce the vibration.

2. The system of claim 1, wherein the dampener is selected from the group consisting of an elastic spring, a sponge, an air cushion, a rubber cushion, a bellows, an air cylinder damper, and combinations thereof.

3. The system of claim 1, wherein the dampener comprises an elastomer selected from the group consisting of rubber, nitrile rubber, fluororubber, silicon, thermoplastic elastomer (TPE), polyurethane, and combinations thereof.

4. The system of claim 3, wherein the elastomer has a hardness of about 20-70 Shore A.

5. The system of claim 3, wherein the elastomer has a hardness of about 30-60 Shore A.

6. The system of claim 1, wherein the system further comprises a silencer disposed between the dampener and the tip and configured to further reduce the vibration.

7. The system of claim 6, wherein the vibration is reduced to a force of about 0-20 gf.

8. The system of claim 1, wherein the piston comprises a longitudinal central axis;
an end wall facing the end wall of the cylinder; a circumferential wall extending from the end wall along the longitudinal central axis, wherein a rack extending along the longitudinal central axis is formed on an outer surface of the circumferential wall of the piston, wherein the driving unit comprises a sector gear having teeth only in a certain angle range, which meshes with the rack of the piston and drives it linearly, wherein the combination of the sector gear and the rack forms the elastic energy release mechanism.

9. The system of claim 8, wherein the driving unit further comprises:
an electrical power supply; and
an electric motor electrically connected to the electric power supply and configured to drive the sector gear rotationally directly or indirectly.

10. The system of claim 1, wherein the elastic member is a coil spring at least partially housed inside the piston.

11. The system of claim 1, wherein the tank is fluidly connected to the throat section of the Venturi tube.

12. The system of claim 1, wherein the formula is a formula for aesthetical purposes.

13. An apparatus for transdermal delivery of an atomized formula, the apparatus comprising:
the system according to claim 1; and
a casing that at least partially houses the system.

14. A kit for transdermal delivery of an atomized formula, the kit comprising:
the apparatus of claim 11; and
a plurality of nozzles, wherein the nozzle is a first nozzle of the plurality of nozzles, and wherein the plurality of nozzles are configured to couple to the apparatus.

15. The kit of claim 14, wherein each nozzle of the plurality of nozzles comprises an outlet hole distinct in size from another nozzle of the plurality of nozzles.

16. The kit of claim 14, wherein each nozzle of the plurality of nozzles is configured to dispense a different formula.

17. The kit of claim 14, wherein the plurality of nozzles is disposable.

18. A method for atomizing and injecting a formula for a transdermal delivery, the method comprising:
filling a tank with the formula, wherein the tank is fluidly connected to a nozzle comprising a tip and a Venturi tube with an internal passage of the Venturi tube via an orifice defined in the Venturi tube, wherein the internal passage comprises a converging section; a diverging section; and a throat section located between the converging section and the diverging section;
displacing a piston in a cylinder in a direction in which a volume of an air intake space in the cylinder increases, wherein the air intake space is defined between the piston and an end wall of the cylinder wherein an outlet hole formed on the end wall of the cylinder is fluidly connected to an inlet of the converging section of the Venturi tube;
deforming, while the piston is being displaced in the direction in which the volume of the air intake space increases, an elastic member that is arranged so as to be deformed according to the displacement of the piston and stores an elastic energy therein;
releasing the elastic energy stored in the elastic member, thereby causing the piston to rush in a direction in which the volume of the air intake space decreases, further causing a vibration from the piston to the Venturi tube;
atomizing and ejecting to outside the formula supplied from the tank into the internal passage of the Venturi tube, by air pushed out from the cylinder through the outlet hole of the end wall of the cylinder with a rush of the piston; and
reducing the vibration with dampener located between the Venturi tube and the tip of the nozzle.

19. The method of claim 18, further comprising further reducing the vibration with a silencer disposed between the dampener and the tip of the nozzle.

20. The method of claim 19, wherein the vibration is reduced to a force of about 0-20 gf.
